(19) United States
(12) Patent Application Publication
(10) Pub. No.: US 2006/0019325 A1
(43) Pub. Date: Jan. 26, 2006

(54) Screening assay

(76) Inventor: Franz Hammerschmid, Vienna (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/524,899

(22) PCT Filed: Sep. 10, 2003
(86) PCT No.: PCT/EP03/10048

§ 371 Date: Feb. 17, 2005

Related U.S. Application Data
(60) Provisional application No. 60/409,732, filed on Sep. 11, 2002.
(60) Provisional application No. 60/409,733, filed on Sep. 11, 2002.

(30) Foreign Application Priority Data
Jan. 10, 2003 (GB) 0300591.5
Jan. 10, 2003 (GB) 0300590.7

(51) Int. Cl. G01N 33/53; C12Q 1/48; G01N 33/537; G01N 33/543

(52) U.S. Cl. 435/7.92; 435/15

(57) ABSTRACT

Screening assays for identifying an agent that modulates the activity of a phosphate catalyzing enzyme.

The present invention relates to a screening assay, e.g. to an assay for identifying an agent that modulates the activity of an enzyme.

In conventional ELISA systems in general either the substance, e.g. an antigen which is to be detected or a specific antibody which binds to the respective antigen, is bound to a solid phase (e.g. microtiter plate) by hydrophobic interactions: the protein interacts with the solid phase, usually a polystyrene surface, at high pH. Although this bond is responsible for all the consecutive steps in the procedure, it remains the weakest bridge to the assay support, the ELISA plate. Strong detergents at higher concentrations such as 0.05% are able to diminish the amount of reagent bound to the plate and can even abolish binding totally. Consecutive steps in ELISA technique, such as e.g. the binding of an antigen to the solid phase-bound antibody and further binding of a second antibody, occur with an affinity (given as K_{d}) of approximately 10⁻¹² to 10⁻¹⁰ mol per liter. One may view this as being similar to an inverse binding cascade from the bottom of the plate to the top, comparing with a pyramide standing on the top.

Another concern is the intramolecular event upon binding. A protein such as a cell-receptor, an enzyme or an antibody behaves very flexible according to its polypeptide structure, which forms a complex architecture in solution. This explains its high specificity and selectivity to the ligands to which they bind in vivo. Enzymatic activities for example may be entirely dependent upon the proper formation of the active site pocket, which itself remains flexible in order to engulf the substrate and release the product.

Most proteins when binding to a given surface react with a dramatic change of their tertiary structure, i.e. they unfold, refold, hide their active site or change their conformation in such a way that their activity towards a given ligand may be altered or even cancelled. In order to circumvent this disadvantage, in conventional ELISA systems a catching antibody is generally used. Said antibody binds to the polystyrene plate and exposes the high affinity hyper-variable region towards the incomig antigen. Said antigen is then detected by a second antibody, which is labelled directly or indirectly (e.g. via biotin/avidin) with an enzyme. This enzyme is able to cleave a chromogenic substrate, which itself is converted from the leucoform to the chromoform and thus visualizes the presence of the antigen in question. But even catching antibodies may affect a given protein and its conformation may be changed. This is demonstrated by many examples of therapeutic antibodies whose mode of action is the blocking of an active site on, or the alteration of a biospecific molecule. We have now found that structural influences or sterical hindrance by the solid phase used in an assay for determining the activity of an enzyme may be avoided and all reaction partners may be provided in solution in defined molar concentrations and therefore determination of enzymatic products may be highly accurate.

In one aspect the present invention provides a method for identifying an agent that modulates the activity of an enzyme comprising:
a) providing a phosphate catalyzing enzyme,
b) providing a peptide,
c) optionally providing a phosphate containing donor,
d) providing a candidate compound,
e) contacting the enzyme of step a) with a peptide of step b) and optionally the phosphate containing donor of step c) in the absence of d) and in the presence of d) for a predetermined period of time so that an enzymatic product can be formed,
f) transferring at least an aliquot of the enzymatic product formed in step e) to a solid phase which is capable to bind to the enzymatic product,
g) detecting the enzymatic product bound to the solid phase,
h) determining whether there is a difference in the amount of enzymatic product in the absence and in the presence of a candidate compound in step e), and
i) choosing an agent that modulates the activity of an enzyme, which method is characterized in that
I) the enzyme is a PAK kinase,
   the peptide comprises the amino acid sequence S-S-L-R-A-S-T,
   the phosphate containing donor of step c) is present in step e), and
   the enzymatic product detected in step g) is the amount of phosphoserine and/or phosphothreonine in the bound peptide; OR
II) the enzyme is CD45 tyrosine phosphatase,
   the peptide comprises the amino acid sequence R-N-Q-E-T-Y-E-T-L-K-H or A-E-N-T-I-T-Y-S-L-L-M-H-P, wherein Y is phosphorylated tyrosine,
   the phosphate containing donor of step c) is absent in step e), and
   the enzymatic product detected in step g) is the amount of phosphotyrosine in the bound peptide.

The principle of this method or assay is as follows: A phosphate catalyzing enzyme has the characteristics that it either transfers phosphate from an external phosphate containing donor to an enzyme specific substrate or that the enzyme dephosphorylates an enzyme specific substrate. The respective product obtained after reaction of an enzyme with the enzyme specific substrate, optionally in the presence of a phosphate containing donor, gives the so called enzymatic product. E.g. in case the enzyme is a PAK kinase, phosphate is transferred from a phosphate containing donor, e.g. ATP, to a PAK specific substrate, e.g. a specific peptide so that a phosphorylated substrate, e.g. peptide, is obtained as the enzymatic product. In case the enzyme is CD45 tyrosine phosphatase, a phosphorylated CD45 tyrosine phosphatase specific substrate, e.g. a specific phosphopeptide, is dephosphorylated so that a dephosphorylated substrate, e.g. peptide, is obtained as the enzymatic product. Preferably the enzyme specific substrate is a peptide, e.g. a pre-labelled peptide. After a predetermined period of time for contacting the enzyme, the substrate, e.g. a peptide according to step b), and in case the enzyme is a PAK kinase also a phosphate containing donor, e.g. ATP, under defined conditions, like e.g. temperature, pH, salt concentration etc., an enzymatic product can be formed. The enzymatic product formed may be either a phosphorylated peptide or a dephopshorylated peptide. The enzymatic reaction is stopped and an aliquot of the enzymatic product formed is transferred to a solid phase, e.g. a streptavidin-coated microtiter plate, where the appropriate peptide, preferably pre-labelled peptide, more preferably the biotinylated peptide, is trapped and the degree (amount) of phosphorylation or dephosphorylation is quantified by means for detecting the enzymatic product formed.

The whole reaction of the enzyme with its reaction partners should occur such, that all molecules are able to float freely in solution and may thus not be influenced by the structural modifications of solid phases. They receive their conformation by the given pH and salt concentration and exert their maximum binding or reaction affinity. Because all the reaction components are provided in solution and the whole reaction is carried out in solution, the exact amounts (concentrations) of all the reaction components are known. The time period which allows measureable formation of an enzymatic product is selected according to the results of appropriate equilibrium measurements. The phosphorylated or dephosphorylated peptide (=enzymatic product) formed is separated from the mixture. The peptide therefore is preferably labelled, e.g. biotinylated, prior to the above reaction and after stopping the reaction, an aliquot of the enzymatic product formed is transferred to a solid phase, preferably to a streptavidin-coated microtiter plate, which binds e.g. to the label of the peptide.

The solid phase is e.g. a plastic plate like a polystyrene or polyvinyl plate, esp. a microtiter plate. Also microbeads can be used as a solid phase, e.g. coated microbeads. The coating used for the solid phase depends e.g. on the label used for the peptide. The material of the coating should be able to form a complex with the label used for the peptide, e.g. the material used for the coating includes streptavidin which is covalently bound to a solid support. The label used for the peptide includes biotin.

The peptide, serving as a specific substrate for a PAK kinase, e.g. a PAK-2 kinase, comprises the amino acid sequence S-S-L-R-A-S-T. Preferably the peptide has the amino acid sequence A-K-R-R-R-L-S-S-L-R-A-S-T-S-K-S. Preferably the peptide is a labelled peptide, e.g. a biotinylated peptide.

The phosphotyrosine containing peptide comprises an amino acid sequence with specificity for CD45 tyrosine phosphatase. Preferably the peptide comprises the amino acid sequence R-N-Q-E-T-Y-E-T-L-K-H or A-E-N-T-I-T-Y-S-L-L-M-H-P, wherein Y is phosphorylated tyrosine.

In another aspect of the present invention the enzymatic product is a phosphorylated serine and/or a phosphorylated threonine containing peptide, which is bound to the solid phase, and the amount of phosphoserine and/or phosphpthreonine in the peptide is detected with an antibody selected from the group consisting of phosphoserine recognizing antibody, phosphothreonine recognizing antibody and phosphoserine and phosphothreonine recognizing antibody.

In a further aspect of the present invention the enzymatic product is a phosphotyrosine containing peptide, which is bound to the solid phase, and the amount of phosphotyrosine in the peptide is detected with an antibody recognizing phosphotyrosine (anti-phosphotyrosine antibody).

Each of the antibodies can itself bear a label and can be detected directly or can bear no label. The label includes one as conventional, e.g. an enzyme like horse radish peroxidase (HRP) or peroxidase (POD) or a fluorescent molecule, e.g. a fluorescent dye. The label bearing antibody may be detected according to methods as conventional, e.g. via fluorescence measurement or enzyme detection methods. An antibody bearing no label may be detected e.g. indirectly e.g. by use of a tandem system of a first antibody recognizing phosphoserine or phosphothreonine or phosphotyrosine and a second antibody specifically recognizing the first antibody, which second antibodies bears a label, e.g. an enzyme or fluorescence label, and may be detected according to methods as conventional, e.g. as indicated herein.

A candidate compound is a compound which may modulate the activity of an enzyme, e.g. a PAK kinase or CD45 tyrosine phosphatase, and includes compound(s)(libraries) from which its influence on the enzyme can be determined. Compound (libraries) include for example oligopeptides, polypeptides, proteins, antibodies, mimetics, small molecules, e.g. low molecular weight compounds (LMW's).

In case that a candidate compound moldulates, e.g. inhibits, a PAK kinase activity in the sample which is to be analyzed, the phosphate catalyzing (transfer) reaction (=phosphorylation) does not occur or occurs only to a limited/reduced extent. In case a candidate compound modulates, e.g. inhibits, the CD45 tyrosine phosphatase, the phosphatase has no or a limited activity to dephosphorylate the peptide. Therefore all or a smaller amount of phosphotyrosine of the bound peptide will be detected.

By comparison of the amounts of remaining phosphoserine and/or phosphothreonine, and/or phosphotyrosine, respectively, in the absence and in the presence of a candidate compound an appropriate agent may be identified.

An agent is a compound which modulates (e.g. inhibits) the activity of an enzyme, e.g. of a PAK kinase, or of CD45 tyrosine phosphatase, respectively. An agent is one of the chosen candidate compounds and may include oligopeptides, polypeptides, proteins, antibodies, mimetics, small molecules, e.g. low molecular weight compounds (LMW's).

In a preferred aspect of the present invention the binding of e.g. streptavidin to the surface of the solid phase, e.g. a microtiter plate, is made covalently in order to build up an affinity cascade from the bottom of the solid phase to the top of the reactants. For example, a microtiter plate chemically modified with e.g. a N-oxysuccinimide ester coating may be used, which reacts with nucleophiles such as primary amines under formation of a covalent bond. Preferably this bond should have a K_{d} of 10⁻¹⁵ mol per liter or les. For the next step, the labelling, e.g. biotinylation of the peptide, e.g. a chemically modified biotin, such as NHS-LC-Biotin is preferably used, which comprises an extended spacer arm of approximately 22.4 Å in length. This long chain analogue may allow the biotin molecule to reach the deep binding cleft of stretavidin, which cleft is more pronounced as compared to avidin. The long spacer may also reduce steric hindrance associated with the binding of four biotinylated molecules to one streptavidin molecule. The amino acid in the peptide to which biotin is bound is well defined and interference with the ligand may be avoided. The binding affinity of e.g. biotin to streptavidin is also known to be 10⁻¹⁵ moles per liter. Thereby the first two steps of the binding cascade are established and reach from the bottom of the plate to streptavidin to the biotinylated peptide. The affinity constants are decreasing from the bottom to the top. At this stage the reactants, which have found their partners in solution under defined conditions, are trapped by means of e.g. the streptavidin coated plate and the bound peptide is then detected, e.g. with an appropriate enzyme-labelled antibody, e.g. an POD-labelled antibody against the phosphoserine and/or phosphothreonine containing peptide or with a tandem antibody system, e.g. as appropriate, such as indicated herein.

Some of the advantages of the method (assay) according to the present invention in comparison to standard ELISA systems where the enzyme or the peptide will first be attached to the solid support with e.g. either a catching antibody or by high pH (9.6) include the following:
a) structural influence or sterical hindrance by the supporting solid phase of the catching antibody in standard sandwich-ELISA may be avoided,
b) treatment of the enzyme or the peptide at alkaline pH may be avoided,
c) the reaction partners may be provided in solution in defined molar concentrations,
d) high specificity for an enzyme because of the peptides used,
e) reduced working steps, and
f) easy handling by e.g. robotics.

In another aspect the present invention provides a method for identifying an agent that modulates the activity of a PAK kinase comprising
a) providing a PAK kinase,
b) providing a peptide comprising the amino acid sequence S-S-L-R-A-S-T, preferably a labelled peptide, e.g. a biotinylated peptide,
c) providing a phosphate containing donor, pref. ATP,
d) providing a candidate compound,
e) contacting the components of steps a), b) and c) in the absence of d) and in the presence of d) for a predetermined period of time so that an enzymatic product can be formed,
f) transferring at least an aliquot of the enzymatic product formed in step e) to a solid phase which is capable to bind the enzymatic product, preferably a solid phase coated with a substrate which is able to bind to the label of the enzymatic product, e.g. a streptavidin-coated solid phase for a biotinylated enzymatic product,
g) determining the amount of the phosphoserine and/or phosphothreonine in the bound enzymatic product,
h) determining whether there is a difference in the amount of phosphoserine and/or phosphothreonine in the absence and in the presence of a candidate compound in step e), and
i) choosing an agent that modulates the activity of a PAK kinase.

In another aspect the present invention provides a method for identifying an agent that modulates the activity of CD45 tyrosine phosphatase comprising:
a) providing CD45 tyrosine phosphatase,
b) providing a peptide comprising the amino acid sequence R-N-Q-E-T-Y-E-T-L-K-H or A-E-N-T-I-T-Y-S-L-L-M-H-P, wherein Y is phosphorylated tyrosine, preferably a labelled peptide, e.g. a biotinylated peptide,
c) providing a candidate compound,
d) contacting the CD45 tyrosine phosphatase of a) with the peptide of b) in the absence of c) and in the presence of c) for a predetermined period of time so that an enzymatic product can be formed,
e) transferring at least an aliquot of the enzymatic product formed in d) to a solid phase which is capable to bind to the enzymatic product, preferably a solid phase coated with a substrate which is able to bind to the label of the enzymatic product, e.g. a streptavidin-coated solid phase for a biotinylated enzymatic product,
f) determining the amount of phosphotyrosine in the bound enzymatic product of step e), and g) determining whether there is a difference in the amount of phosphotyrosine in the absence and in the presence of a candidate compound in step d), and
h) choosing an agent that modulates the activity of CD45 tyrosine phosphatase.

In another aspect the present invention provides a kit for identifying an agent that modulates the activity of a PAK kinase comprising as components
a) a PAK kinase,
b) a peptide comprising the amino acid sequence S-S-L-R-A-S-T, preferably a labelled peptide, e.g. as defined above,
c) a phosphate containing donor, preferably ATP,
d) means for detecting phosphoserine and/or phosphotyrosine, and
e) optionally a solid phase, preferably coated with a substrate which is able to bind to the label of the peptide.

In another aspect the present invention provides a kit for identifying an agent that modulates the activity of CD45 tyrosine phosphatase comprising as components
a) a CD45 tyrosine phosphatase,
b) a peptide comprising the amino acid sequence R-N-Q-E-T-Y-E-T-L-K-H or A-E-N-T-I-T-Y-S-L-L-M-H-P, wherein Y is phosphorylated tyrosine, preferably a labelled peptide, e.g. a biotinylated peptide,
c) means for detecting phosphotyrosine, and
d) optionally a solid phase, preferably coated with a substrate which is able to bind to the label of the peptide.

Means for detecting phosphoserine and/or phosphotyrosine include e.g. antibodies, preferably an antibody selected from the group consisting of phosphoserine recognizing antibody, phosphothreonine recognizing antibody and phosphoserine and phosphothreonine recognizing antibody. Preferably a phosphoserine recognizing antibody and a phosphothreonine recognizing antibody are used.

Means for detecting phosphotyrosine include e.g. antibodies, preferably a phosphotyrosine recognizing antibody. Preferably the antibody bears a label, e.g. an enzyme or a fluorescent label.

The antibody(ies) can be labelled or not and detection is carried out as indicated herein. Said kits may further comprise a substantial component, e.g. Including an appropriate environment of a sample to be tested and, e.g. appropriate means to determine the effect of a candidate compound in a sample to be tested.

In a further aspect the present invention provides a method for differentiating whether an agent is capable to modulate the serine specific activity of a PAK kinase or the threonine specific activity of a PAK kinase, or both, comprising
a) providing a PAK kinase,
b) providing a peptide comprising the amino acid sequence S-S-L-R-A-S-T,
c) providing a phosphate containing donor,
d) providing a candidate compound,
e) contacting the enzyme of step a) with a peptide of step b) and the phosphate containing donor of step c) in the absence of d) and in the presence of d) for a predetermined period of time so that a phosphoserine and/or phosphothreonine containing peptide can be formed,
f) transferring at least an aliquot of the peptide formed in step e) to a solid phase which is capable to bind to the peptide,
g) detecting the amount of phosphoserine and phosphothreonine in the bound peptide by use of at least 2 different antibodies selected from the group consisting of phosphoserine recognizing antibody, phosphothreonine recognizing antibody and phosphoserine and phosphothreonine recognizing antibody, and
h) determining if the agent modulates the serine specific activity of a PAK kinase, or the threonine specific activity of a PAK kinase, respectively, or both.

In a preferred aspect the antibodies bear a different label and the amount of phosphoserine and phosphothreonine is determined simultaneously. E.g. one of the antibodies is a phosphoserine recognizing antibody and the label is a fluorescence label and the other antibody is phosphothreonine recognizing antibody and the label is an enzyme, e.g. POD. Preferably the antibodies are differently labeled, e.g. bear 2 different fluorescent labels, such as conventional, e.g. Cy5, TAMRA or PODIPY/see e.g. Molecular Probes catalogue). An antibody may also be derived from different species, e.g. it may be a mouse or a rabbit antibody.

In another aspect the present invention provides the use of a peptide comprising the amino acid sequence S-S-L-R-A-S-T, preferably a peptide having the sequence A-K-R-R-R-L-S-S-L-R-A-S-T-S-K-S, for identifying an agent that modulates the activity of a PAK kinase.

In another aspect the present invention provides the use of a peptide comprising the amino acid sequence S-S-L-R-A-S-T, preferably a peptide having the sequence A-K-R-R-R-L-S-S-L-R-A-S-T-S-K-S, for differentiating between an agent that modulates the serine specific activity of a PAK kinase and/or the threonine specific activity of a PAK kinase and/or both.

In another aspect the present invention provides the use of a peptide comprising the amino acid sequence R-N-Q-E-T-Y-E-T-L-K-H or A-E-N-T-I-T-Y-S-L-L-M-H-P, wherein Y is phosphorylated tyrosine, for identifying an agent that modulates the activity of CD45 tyrosine phophatase.

An agent of the present invention may exhibit pharmacological activity and is therefore useful as a pharmaceutical, e.g. for modulation of the immune response. An agent of the present invention may show therapeutic activity and may therefore be used for treatment of e.g. autoimmune diseases. An agent of the present invention for treatment includes one or more, preferably one, agent of the present invention, e.g. a combination of two or more agents of the present invention.

In another aspect the present invention provides an agent for use as a pharmaceutical.

In a further aspect the present invention provides a pharmaceutical composition comprising an agent identified by a method according to the present invention as an active ingredient in association with at least one pharmaceutical excipient.

In another aspect the present invention provides the use of an agent of the present invention for the manufacture of a medicament, e.g. a pharmaceutical composition, for the treatment of autoimmune diseases or inflammatory diseases.

The pharmaceutical compositions according to the present invention may be used for the treatment of a disorder having an etiology associated with the production of a substance, e.g. an inflammatory acting (causing/enhancing) substance, selected from the group consisting of cytokine, growth factor, proto-oncogene or viral protein. Preferably said substance is selected from the group consisting of IL-1, IL-2, IL-3, IL4, IL-8, GM-CSF, TNF-α, VEGF, AT-R1, Cox-2, c-fos and c-myc. Treatment includes treatment and prophylaxis. For such treatment, the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 1.0 g, of an agent of the present invention; conveniently administered, for example, in divided doses up to four times a day. Ap agent of the present invention may be administered by any conventional route, for example enterally, e.g. including nasal, buccal, rectal, oral administration; parenterally, e.g. including intravenous, intramuscular, subcutanous administration; or topically; e.g. including epicutaneous, intranasal, intratracheal administration; e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories. An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition comprising an agent of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such compositions may be manufactured according, e.g. analogously to a method as conventional, e.g. by mixing, granulating, coating, dissolving or lyophilizing processes. Unit dosage forms may contain, for example, from about 0.5 mg to about 2000 mg, such as 1 mg to about 500 mg, e.g. 0.00625 mg/kg to about 12.5 mg/kg.

In another aspect the present invention provides an assay for identifying an agent that modulates the activity of a PAK kinase comprising the steps of:
a) providing a peptide comprising the amino acid sequence S-S-L-R-A-S-T, preferably a labelled peptide, e.g. a biotinylated peptide, in a defined amount,
b) providing a PAK kinase in a defined amount,
c) providing a phosphate containing substance, pref. ATP,
d) contacting the components of step a), b) and c) in the absence and in the presence of a candidate compound which is expected to modulate the activity of a PAK kinase for a sufficient period of time so that a reaction mixture is formed and reacting for a predetermined period of time,
e) transferring at least an aliquot of the reaction mixture formed in step d) to a solid phase, preferably a solid phase coated with a substrate which is able to bind to the label of the peptide, e.g. a streptavidin-coated solid phase for a biotinylated peptide,
f) detecting the amount of peptide bound to the solid phase of step e) and determining whether there is a difference in the amount of bound peptide in case a candidate compound was present or absent in the reaction mixture, and
g) choosing an agent that modulates the activity of a PAK kinase.

In another aspect the present invention provides an assay for identifying an agent that modulates the activity of a CD45 tyrosine phosphatase comprising the steps of:
a) providing a peptide comprising the amino acid sequence R-N-Q-E-T-Y-E-T-L-K-H or A-E-N-T-I-T-Y-S-L-L-M-H-P, wherein Y is phosphorylated tyrosine, preferably a labelled peptide, e.g. a biotinylated peptide, in a defined amount,
b) providing a CD45 tyrosine phosphatase in a defined amount,
c) contacting the peptide of step a) with the CD45 tyrosine phosphatase of step b) in the absence and in the presence of a candidate compound which might modulate the activity of a CD45 tyrosine phosphatase for a sufficient period of time so that a reaction mixture is formed and reacting for a predetermined period of time,
d) transferring at least an aliquot of the reaction mixture formed in step c) to a solid phase, preferably a solid phase coated with a substrate which is able to bind to the label of the peptide, e.g. a streptavidin-coated solid phase for a biotinylated peptide,
e) detecting the amount of peptide bound to the solid phase of step d) and determining whether there is a difference in the amount of bound phosphorylated peptide in case a candidate compound was present or absent in the reaction mixture, and
f) choosing an agent from said candidate compound detected in step e), e.g. for use as a pharmaceutical.

In the following examples all temperatures are in degree centigrade (° C.) and are uncorrected.

The following ABBREVIATIONS are used:
Ahx 6-aminohexanoic acid
ATP adenosine triphosphate
BSA bovine serum albumin
DEAE diethylaminoethyl
DTT dithiothreitol
DMF N,N′-dimethylformamide
DMSO dimethylsulfoxide
EDTA ethylene diamine tetraacetic acid
ELISA enzyme-linked immunosorbent assay
FCS fetal calf serum
FPLC forced pressure liquid chromatography
IPTG isopropyl-β D-thiogalactopyranosid
LB AMP 100 LURIA-BERTANI medium with 100 μg/ml ampicillin
LC long chain spacer arm
mAb monoclonal antibody
OD optical density
PBS phosphate-buffered saline
PBSdef. NaCl 8 g/l; KH₂PO₄ 0.2 g/l; KCl 0.2 g/l; Na₂HPO₄.2aq 1.44 g/l; pH 7.2
PBST PBS+0.05% Tween 20
POD horse radish peroxidase
pNPP p-nitrophenylphosphate
PTPase phosphotyrosine phosphatase
rpm rounds per minute
RT room temperature,
TFA trifluoroacetic acid
TMB teramethylbentidine=3,3′,5,5′-tetrameathyl-benzidine dihydrochloride

### EXAMPLES

### Example 1

### PAK Kinase Assay

a.) Preparation of Covalently-Bound Streptavidin Microtiter Plates

6 μl of streptavidin solution are added to 12 ml of PBSdef. of pH 9 and stirred. 100 μl of the solution obtained are pipetted into each well of a Costar Amine plate™, which is taken directly from 40 storage and dismanteled from its protective envelope prior to pipetting. The whole procedure is performed in the dark and the plate is kept in a light protective aluminium foil at RT. Consecutive steps are carried out under conditions as indicated herein. The plate is washed 5 times with PBST and tapped onto a paper towel in order to free the plate from residual moisture. 360 μl of 0.5 M Tris-Cl pH 8 are added and incubation is done at RT. After washing 5 times as indicated herein, 200 μl of blocking solution are added into each well and the well obtained is kept at RT for 30 minutes. The plate is washed once more and stored in a sealed plastic box at 40.

b.) Biotinylation of the Peptide

A peptide comprising the amino acid sequence S-S-L-R-A-S-T, e.g. a peptide with the amino acid sequence A-K-R-R-R-L-S-S-L-R-A-S-T-S-K-S is used as a substrate. The peptide is dissolved in either DMF or DMSO and is kept in a light protected vial. A solution of Immunopure NHS-LC-Biotin II (Pierce) is prepared in above solutes and kept under light protection. The biotin solution is added dropwise to the peptide containing vial at RT. The reaction is stopped by adding 1 M Tris-Cl pH 8 and tumbled. The reaction mixture obtained is transferred into Eppendorf vials and centrifuged in order to clarify the solution of biotinylated peptide. The supernatant obtained is subjected to reversed phase chromatography or size exclusion chromatography.

c.) Assay Procedure (Key Steps)

a) Mix biotinylated peptide, ATP, PAK-kinase and optionally a candidate compound
b) Incubate so that a reaction mixture is formed
c) Provide plates with covalently coupled streptavidin (=SA-plates) and incubate
d) Transfer an aliquot of the reaction mixture formed in step b) onto SA-plate
e) Wash the plate free of unbound substances
f) Add detecting antibody (=POD-linked anti-phosphoserine antibody and/or POD-linked anti-phosphothreonine antibody and/or anti-pgosphoserine-phosphotyrosine antibody, and optionally a labelled secondary antobidy recognizing the first unlabelled antibody, and incubate
g) Wash and develop enzyme substrate, e.g. peroxidase substrate, and
h) Determine the extinction at defined wavelength, e.g. at 450/690 nm to quantify the enzymatic reaction and thus determine the amount of phosphorylated (phosphorylation degree) serine and/or threonine.

### EXAMPLE 2

### CD45 Tyrosine Phosphatase Assay

a) Preparation of LCA D1D2 (=CD45 Tyrosine) Phosphatase

The pT7 LCA D1 D2 plasmid encodes' both the D1 and D2 cytoplasmic domains of the human LCA PTPase. The LCA-D1D2 PTPase contains 8 amino acids (M-A-R-I-R-A-R-G) derived from the pT7-7 vector at the N terminus and residues 584 through the most C-terminal residue (position 1281) of the human LCA protein. (see e.g. J. Biol. Chem. Vol. 267, pp12356-12363).

b) Overproduction of CD45 Tyrosine Phosphatase

In order to overproduce this enzyme E. coli BL21 is transformed with the plasmid pT7-LCA-D1D2, which carries an integrated copy of the T7 RNA polymerase gene under the control of the inducible lacUV5 promoter. The transformed E. coli BL21 are assayed for the presence of the plasmid (mini-preps) and for the expression of the respective protein. Stocks are prepared from the mid-log-phase by freezing the bacterial suspension in 50% glycerol at −190° and are stored at −80°.

c) Purification of the CD45 Tyrosine Phosphatase

A 1 ml aliquote from the stock-culture is thawed and serves as inocculum for a 200 ml LB AMP 100 [500 ml Erienmeyer flask]. The culture is agitated at 200 rpm and 37° to an E₆₀₀=0.7, where 8 times 33 ml are transferred into 8 times 1 liter LB Amp 100. This culture is grown under the same conditions until an E₆₀₀ of 0.7 is achieved. At this point 10 ml of a sterile stock solution of 100 mM IPTG are added in order to adjust the bacterial culture to 1 mM IPTG. The temperature is lowered to 28° and the agitation at 200 rpm is continued for three more hours. Cells are harvested by centrifugation at 18.200×g at 4°. The cell mass is resuspended in 30 ml of buffer A (33 mM Tris-HCl pH 8; 25 mM EDTA; 10 mM 2-mercapto-ethanol) to give a concentration of approx. 130 mg weight/ml. In order to provide a rapid heat transfer the suspension is divided into 30 ml aliquotes and kept in 50 ml vials. The vials are submersed into liquid nitrogen until equilibrium is reached and afterwards they are submersed into a 37° waterbath until the content is liquified. 2 ml of a 6 mg/ml solution of Lysozyme (Boehringer) are added and the vials are agitated on a reciprocate shaker at 50 rpm at RT. 1 ml of Trasylol™ (Bayer), 30 μl of Leupeptin™ (Boehringer Mannheim, 50 mg/ml), 2 ml of 500 mM MgCl₂, a few grains of DNAse I (Boehringer) are added to each vial and agitated as indicated herein. The reaction is stopped by addition of 2 ml of 500 mM EDTA, 300 μl of Triton-X100 are added and the solubilization procedure is carried on during agitation. The extract is clarified by centrifugation at 120.000×g at 4°. All supernatants are pooled and adjusted to 35% (NH₄)₂SO₄ under slow addition of the powder under stirring at RT and subsequent stirring at 4°. The precipitate obtained is collected by centrifugation as indicated herein and the supernatant is adjusted for 60% (NH₄)₂SO₄ under conditions indicated herein. The precipitate obtained containing the product is harvested by centrifugation as indicated herein, where the pellet obtained is dissolved by 10 ml of buffer B (=33 mM Tris-HCl pH 8.5; 2.5 mM EDTA; 10 mM 2-mercaptoethanol) plus 3 ml of Trasylol™ and 0.5 ml of Leupeptin™. The sample obtained is dialyzed at two consecutive steps at 4°: once against 2 liters of buffer B for one hour and then against 5 liters of buffer B. The first chromatographic purification is done with a DEAE Sepharose Fast Flow™ column equilibrated in buffer B. All purifications are carried out at 4°. The gradient is formed against buffer B containing 1 M NaCl. (flow rate 2 ml/min, gradient 0-30%: 0.1%/min; gradient 30-100%: 1%/min; sample application and wash: 12 ml/fraction; gradient: 4 ml/fraction). The fractions obtained are analyzed for phosphatase activity with RNQETY(PO₃H₂)ETLKH and pNPP as substrates (see h) assays). The fractions obtained containing the specific phosphatase activity are pooled and concentrated by ultrafiltration (Amicon PM30/76) under addition of 2 ml of Trasylol™, 1 ml of Leupeptin™ and 10 ml of glycerol to approximately 20 ml. The sample obtained is further purified over a Blue Sepharose™ column equilibrated in buffer B containing 10% glycerol; pH 7.4. The gradient is formed against buffer B containing 1 M NaCl; 10% glycerol; pH 9.4. (flow rate 2 ml/min, gradient 0-100%: 0.33%/min; sample application and wash: 12 ml/fraction; gradient: 4 ml/fraction). Phosphatase containing fractions are pooled and concentrated by ultrafiltration (Amicon PM30/76), 10 ml of Trasylol™ and 1 ml of Leupeptin™ are added to 11 ml of the fraction obtained. The sample obtained is loaded for consecutive runs at 2.5 ml onto a Superdex 75™ size-exclusion column equilibrated in a buffer mixture of 40% buffer B in buffer A of conditions as indicated herein. The chromatography is done at isocratic conditions and fractions of 2.6 ml are taken. After assaying for phosphatase activity in both ways, active fractions are pooled and concentrated to 12 ml. 1 ml of Leupeptin™, 1 ml of Trasylol™ and 46.5 ml of glycerol are added. The preparation obtained is divided into 30 μl aliquotes which are stored in Micronic Racks (96 vials) at −80°. Final concentrations of such a preparation are the following:

Protein 728 μg/ml, tris 8.9 mM, EDTA 0.7 mM, NaCl 108 mM, 2-mercaptoethanol 2.7 mM, aprotinin 32 μg/ml, Leupeptin™ 1124 μg/ml and glycerol 75%. The preparation obtained is assayed for activity using the gamma-substrate RNQETY(PO₃H₂)ETLKH as a serial dilution of the enzyme. The enzyme obtained shows phosphatase activity.

d) Biotinylation of Peptide Substrates

5 μmol of phosphotyrosine peptide are dissolved in 500 μl of either DMF or DMSO and are kept in an light protected vial. A solution of Immunopure NHS-LC-Biotin II (Pierce) is prepared in above solutes and kept under light protection too. 500 μl of biotin solution are added to the peptide containing vial, which is tumbled at 25 rpm at RT. Another 500 μl of the biotin solution are added and the vial is tumbled for 3 hours under the conditions as. Indicated herein. The reaction is stopped by adding 500 μl of 1 M Tris-Cl pH 8 and tumbled for 30 minutes. The reaction mixture obtained is transferred into Eppendorf vials (500 μl each) and centrifuged at 14.000 rpm in order to clarify the solution of biotinylated peptide. The supernatant obtained is subjected to HPLC purification on Vydac C₁₈ (218 TBP 10 μ 740711) equilibrated to 5% CH₃CN; 0.1% TFA. Peptide is eluted by a gradient toward 100% CH₃CN; 0.1% TFA. (flow rate 2 ml/min; gradient 0-100%: 1%/min, fraction size: 2 ml/fraction). Biotinylated peptide is obtained.

e) Preparation of Covalently Bound Streptavidin Microtiter Plates

6 μl of streptavidin solution are added to 12 ml of PBSdef. of pH 9 and stirred. 100 μl of the solution obtained are pipetted into each well of a Costar Amine plate, which is taken directly from 4° storage and dismanteled from its protective envelope prior to pipetting. The whole procedure is performed in the dark and the plate is kept in a light protective aluminium foil at RT. Consecutive steps are carried out under conditions as Indicated herein. The plate is wshed 5 times with PBST and tapped onto a paper towel in order to free the plate from residual moisture. 360 μl of 0.5 M Tris-Cl pH 8 are added and incubated at RT. After washing 5 times with PBST, 200 μl of blocking solution are added into each well and the well obtained is kept at RT for 30 minutes. The plate obtained is washed once more and stored in a sealed plastic box at 4°. The following peptide substrates are provided:

| | |
|---|---|
| FcεR1γ | RNQETY(PO₃H₂)ETLKH |
| FcεR1γ.biot | biotinyl-6-AhxRNQETY(PO₃H₂)ETLKH |
| ITIM | AENTITY(PO₃H₂)SLLMHP |
| ITIM.biot | biotinyl-6-AhxAENTITY(PO₃H₂)SLLMHP |

f) Assay for the Detection of Both Biotinylation and Function of the Phosphotyrosine Peptide

50 μl of 200 mM NaHCO₃ are pipetted into a U-bottom microtiter plate, 30 μl of fraction aliquotes are added in order to neutralize the TFA and 60 μl of the plate obtained are copied into a microtiter plate covalent coupled with streptavidin and kept at RT. The plate obtained is washed 5 times with PBST and 100 μl of anti-phosphotyrosine antibody 1:2000 in incubation buffer are added. After incubation the plate is washed again as indicated herein and 100 μl of anti-mouse-POD antibody 1:2000 in incubation buffer are added. After subsequent incubation the plate is washed with PBST as indicated herein and 100 μl of TMB-substrate are added. After incubation at RT the reaction is stopped by adding 4N H₂SO₄ and the plate is read at 450/690 nm. By this assay only those fractions give positive results which are tyrosine phosphorylated, not aggregated and properly biotinylated.

g) Cell-Experiments

1. Purified naïve and effector T cells (10⁸ ml) are stimulated at 37° with F(ab′)₂ (12 μg/ml) or intact anti-mouse IgG antibody (20 μg/ml). Unstimulated and stimulated cells are lysed in 1% NP-40 (Sigma) lysis buffer (=1% NP-40; 10 mM Tris-Cl pH 7.4; 150 mM NaCl; 400 μM EDTA; 1.4 ml/liter Trasylol™) and spun at 14.000 rpm in an Eppendorf microfuge to remove detergent-insoluble material. A serial dilution of the cell lysate obtained in buffer (160 μl) is incubated with 80 μl of biotinylated peptide substrate (3 nM) at 370. A 100 μl aliquote is transferred to a plate coated with Streptavidin and incubated at 40 (see e.g. Science, 268,(1995), p293-297).

2. Purified naïve and effector T cells (10⁸/ml) are treated with inhibitors at different concentrations and different times of incubation. 240 μl of the respective sample are pipetted into the top-row of a U-bottom microtiter plate A1-A12 where 60 μl of a 5-fold concentrated lysis buffer (see above) are already placed. The cells are lysed by mixing with a 12-channel pipettor. 100 μl of the lysate obtained are transferred into 200 μl of substrate at 1.5 nM concentration (row B1-B12), mixed and further diluted into substrate at 1 nM concentration (row C1-C12) by transferring 100 μl into 200 μl of substrate. These steps are consequently repeated from row C to row H. The plate is incubated at 37°, the reaction is stopped with 25 mM sodium-meta-vanadate and 100 μl of the plate are copied into a streptavidin-microtiter-plate (SA-covalent). CD45 tyrosine phosphatase activity is shown in determining the amount of phosphotyrosine.

h) Assays

1. Phosphatase Assay During Purification Procedure for LCA D1D2

The assay buffer described here is identical with the system for peptide substrates, namely: 100 mM Tris, 5 mM EDTA, 10 mM DTT, 50 mM NaCl, 50 μg BSA/ml, 1.4 ml Trasylol™/liter, 5% glycerol at pH 7.2. For column fraction assays this buffer system is adjusted to 10 mM pNPP. 20 μl fraction aliquotes are pipetted into a microtiter plate (flat bottom), 100 μl of substrate are added and the plate is incubated at RT. The reaction is stopped by adding 100 μl of 132 mM NaOH (60 mM final concentration) and the plate is read at 405 nm. For specific assay of column fractions 10 μl of sample is placed into each well of a U-bottom microtiter plate and 40 μl of assay buffer are added. 50 μl of the gamma substrate are added and the plate is incubated at 37°. 100 μl of aliquote are transferred to a streptavidin-plate and processed as indicated herein. The activity of the CD45 tyrosine phosphatase is shown.

2. Specific CD45 Tyrosine Phosphatase Assays

All substances are distributed into microtiter plates for testing in solutions of 10 mM NaCl, 50% DMSO at a concentration of 200 μg/ml or 500 μM. The solutions obtained in conical bottom plates are diluted once more 1:3.3 (20 μl of substance plus 46 μl of assay buffer) into round bottom plates in order to give 66 μl 150 mM substance in 15% DMSO. The plates obtained are adjusted in position A4-A12 by emptying the wells by suction (the whole line A1-A3 is dedicated for medium for medium references=High control) and 1 M HCl is pipetted into well A9-A12 in order to give the Low control. Sodium-vanadate at 500 mM is adjusted to pH 7.2 and diluted to 40, 20, 10, 5, 2.5 mM and pipetted into A4-A8 in order to give the calibration curve for sensitivity. The plate obtained is assigned as dilution plate. The working solution for the biotinylated peptide substrate is prepared by diluting the stock solution in assay buffer to a final concentration of 3 nM. 50 μl of the solution obtained are distributed to each well of a new round bottom plate (incubation plate) and 50 μl of the respective dilution plate are transferred to this plate and mixed with the peptide substrate for five times. The plate is kept at RT, 50 μl of an adequate dilution of CD45 tyrosine phosphatase in assay buffer (4.8 μl/ml) are added to each well of the incubation plate and mixed five times. The respective concentration are as follows: 5% DMSO, 50 μM substance, 300 mM HCl, nM peptide substrate, 13.3; 6.7; 3.3; 1.7; and 0.8 mM sodium vanadate. The plate is sealed with an adhesive foil and incubated for one hour at 37°. The reaction is stopped by adding 50 μl of 25 mM sodium-meta-vanadate and 100 μl are transferred from the incubation plate into the streptavidin-coated plate, which is incubated at RT. After washing 5 times with PBST, 100 μl of a mixture of anti-tyrosine-phosphate antibody dilution 1:2000 and anti-mouse-POD labelled antibody dilution 1:500 in incubation buffer are added to each well and the plate is incubated again at RT. The plate obtained is washed again 5 times and 100 μl of TMB-substrate are added. After incubation at RT, the reaction is stopped by adding 100 μl 4 N H₂SO₄ and the plate is read at 450 nm/690 nm. CD45 tyrosine phosphazase activity is shown.

1. A method for identifying an agent that modulates the activity of an enzyme comprising
a) providing a phosphate catalyzing enzyme,
b) providing a peptide,
c) optionally providing a phosphate containing donor,
d) providing a candidate compound,
e) contacting the enzyme of step a) with a peptide of step b) and optionally the phosphate containing donor of step c) in the absence of d) and in the presence of d) for a predetermined period of time so that an enzymatic product can be formed,
f) transferring at least an aliquot of the enzymatic product formed in step e) to a solid phase which is capable to bind to the peptide in the enzymatic product,
g) detecting the enzymatic product bound to the solid phase,
h) determining whether there is a difference in the amount of enzymatic product in the absence and in the presence of a candidate compound in step e), and
i) choosing an agent that modulates the activity of an enzyme, which method is characterized in that
I) the enzyme is a PAK kinase,
the peptide comprises the amino acid sequence S-S-L-R-A-S-T,
the phosphate containing donor of step c) is present in step e), and
the enzymatic product detected in step g) is the amount of phosphoserine and/or phosphothreonine in the bound peptide; OR
II) the enzyme is CD45 tyrosine phosphatase,
the peptide comprises the amino acid sequence R-N-Q-E-T-Y-E-T-L-K-H or A-E-N-T-I-T-Y-S-L-L-M-H-P, wherein Y is phosphorylated tyrosine,
the phosphate containing donor of step c) is absent in step e), and
the enzymatic product detected in step g) is the amount of phosphotyrosine in the bound peptide.

2. A method according to claim 1 wherein the amount of phosphoserine and/or phosphothreonine is determined with an antibody selected from the group consisting of phosphoserine recognizing antibody, phosphothreonine recognizing antibody and phosphoserine and phosphothreonine recognizing antibody and the amount of phosphotyrosine is determined with a phosphotyrosine recognizing antibody.

3. A method according to claim 1 wherein the enzyme is a PAK kinase and peptide has the amino acid sequence A-K-R-R-R-L-S-S-L-R-A-S-T-S-K-S.

4. A kit for identifying an agent that modulates the activity of a PAK kinase comprising as components
a) a PAK kinase,
b) a peptide comprising the amino acid sequence S-S-L-R-A-S-T,
c) a phosphate containing donor,
d) means for detecting phosphoserine and/or phosphothreonine, and
e) optionally a solid phase.

5. A kit for identifying an agent that modulates the activity of CD45 tyrosine phosphatase comprising as components
a) a CD45 tyrosine phosphatase,
b) a peptide comprising the amino acid sequence R-N-Q-E-T-Y-E-T-L-K-H or A-E-N-T-I-T-Y-S-L-L-M-H-P, wherein Y is phosphorylated tyrosine,
c) means for detecting phosphotyrosine, and
d) optionally a solid phase.

6. A method for differentiating whether an agent is capable to modulate the serine specific activity of a PAK kinase or the threonine specific activity of a PAK kinase or both comprising
a) providing a PAK kinase,
b) providing a peptide comprising the amino acid sequence S-S-L-R-A-S-T,
c) providing a phosphate containing donor,
d) providing a candidate compound,
e) contacting the enzyme of step a) with a peptide of step b) and the phosphate containing donor of step c) in the absence of d) and in the presence of d) for a predetermined period of time so that a phosphoserine and/or phosphothreonine containing peptide can be formed,
f) transferring at least an aliquot of the peptide formed in step e) to a solid phase which is capable to bind to the peptide,
g) detecting the amount of phosphoserine and phosphothreonine in the bound peptide by use of at least 2 different antibodies selected from the group consisting of phosphoserine recognizing antibody, phosphothreonine recognizing antibody and phosphoserine and phosphothreonine recognizing antibody, and
h) determining if the agent modulates the serine specific activity of a PAK kinase, or the threonine specific activity of a PAK kinase, respectively, or both.

7. A method according to claim 6 wherein the antibodies bear a different label and the amount of phosphoserine and phosphothreonine is determined simultaneously.

8. The use of a peptide comprising the amino acid sequence S-S-L-R-A-S-T for identifying an agent that modulates the activity of a PAK kinase.

9. The use of a peptide comprising the amino acid sequence S-S-L-R-A-S-T for differentiating between an agent that is capable to modulate the serine specific activity of a PAK kinase or the threonine specific activity of a PAK kinase or both.
